(19) United States
(12) Patent Application Publication
(10) Pub. No.: US 2007/0233077 A1
(43) Pub. Date: Oct. 4, 2007

(54) Dynamic intervertebral spacer assembly

(76) Inventor: Farid B. Khalili, Briar Cliff Manor, NY (US)

Correspondence Address: Lawrence Cruz, 12 Goodhill Rd., Bethel, CT, 06801, US

(21) Appl. No.: 11/395,945

(22) Filed: Mar. 31, 2006

(51) Int. Cl. A61F 2/30

(52) U.S. Cl. 606/61

(57) ABSTRACT

An implantable spacer body (44) configured to be positioned between the spinous process portions of adjacent vertebrae in such a manner that the spinous process of the superior vertebra rests on and is supported on the spacer body. A pair of extension arms (54, 56) extend from each side of the spacer body and are adapted to be fixed to bone fasteners (86) which engage a region of the inferior vertebral body in locations other than the spinous process, preferably engaging the pedicle regions of the inferior vertebra. Alternatively, the extension arms can be fixed to spinal rods (90, 92) that are fastened to multiple vertebrae in a typical spinal rod assembly.

### RELATED APPLICATIONS

None.

### TECHNICAL FIELD

The present invention relates to implantable spacers for implantation between two vertebral segments and, more particularly, to a spacer that permits controlled movement while providing support between adjacent vertebrae with optimal load distribution.

### BACKGROUND OF THE INVENTION

In spinal orthopedic surgical techniques and procedures, it is common to replace and/or supplement injured, diseased, worn or otherwise deficient components of the spine including vertebrae and disc tissue between vertebrae. While in some cases, fusion is desired, in other cases it is desirable to provide a dynamic intervertebral spacer assembly which permits controlled movement along with support for sufficient vertebral spacing and stability.

The various known systems for allowing controlled movement of spinal reconstructions have shortcomings. Such shortcomings include: overloading a specific area such as a disc or disc region by concentrating load which by nature is meant to be distributed among additional vertebral components; permitting over-bending of a repaired spinal section so as to increase risk of over-stretching and injury; requiring highly invasive procedures for installation; lacking versatility to adapt to varying sizes and configurations; and lacking overall stability.

### OBJECTS OF THE INVENTION

It is an object of the present invention to provide an implantable, dynamic intervertebral spacer assembly that overcomes the above-mentioned shortcomings and that achieves additional, inherent objectives apparent from the description set forth below.

### SUMMARY OF THE INVENTION

The present invention comprises an implantable spacer body configured to be positioned between the spinous process portions of adjacent vertebrae in such a manner that the spinous process of the superior vertebra rests on and is supported on the spacer body. A pair of extension arms extend from each side of the spacer body and are adapted to be fixed to bone fasteners which engage a region of the inferior vertebral body in locations other than the spinous process, preferably engaging the pedicle regions of the inferior vertebra. Alternatively, the extension arms can be fixed to spinal rods that are fastened to multiple vertebrae in a typical spinal rod assembly.

### BRIEF DESCRIPTION OF THE DRAWINGS

FIG. 1 is a diagrammatic sideview of a pair of adjacent vertebrae for use in illustrating application of the present invention.

FIG. 2 is a diagrammatic top view of a vertebra for use in illustrating application of the present invention.

FIG. 3 is a perspective view of a first preferred embodiment of a device according to the present invention.

FIG. 4 is a perspective view of a second preferred embodiment of a device according to the present invention assembly.

FIG. 5 is a diagrammatic top view of an assembly according to the first embodiment of the present invention.

FIG. 6 is a partial, perspective view of a component of the device according to FIG. 3, having a fastener attached thereto.

FIG. 7 is a diagrammatic top view of an assembly according to the first embodiment of the present invention, having a pair of fasteners attached thereto.

FIG. 8 is a diagrammatic end view of the assembly shown in FIG. 7.

FIG. 9 is a diagrammatic end view of an alternative assembly according to the present invention.

### DESCRIPTION OF THE PREFERRED EMBODIMENTS

In describing and illustrating the preferred embodiments, reference is made to the diagrammatic representations of vertebral components shown in FIGS. 1 and 2. A pair of adjacent vertebrae are represented as a superior vertebra (10) and an inferior vertebra (12), described with reference to orientation arrows shown in FIG. 1 as: a superior direction (14), and inferior direction (16), an anterior direction (18), and a posterior direction (20). The first and second vertebrae have various components that are common to different regions of the spine such as cervical, thoracic, and lumbar regions, even though the actual shapes and details of individual vertebra vary among such regions. Despite minor differences, the presently claimed invention has application that is not limited to any one of these regions and has the versatility to be configured in a manner in which it is applicable to any region.

The first vertebra (10) has a body (22) with an upper end face (24), a lower end face (26), a pedicle region (28) on each side, and a spinous process (30). The second vertebra (12) has a body (32) with an upper end face (34), a lower end face (36), a pedicle region (38) on each side, and a spinous process (40). A vertebral space (42) exists between the adjacent vertebrae (10, 12) and is ordinarily occupied by vertebral disc tissue (not shown).

A first embodiment of the present invention is shown in FIG. 3, and comprises a body portion (44) having generally symmetrical and generally parallel disc-shaped sections (46, 48) joined by a middle portion (50) of a smaller diameter than the disc-shaped sections (46, 48) such that the middle portion (50) forms a generally horizontal axis when the disc-shaped portions (46, 48) are aligned with an imaginary vertical axis (52). A first arm (54) and a second arm (56) each extend from a respective one of said disc-shaped sections (46, 48) away from the center of the body portion (44) and, preferably, at an angle (α) with respect to an imaginary horizontal centerline (58) as shown by dotted lines in FIG. 3 extending from a distal end of each arm (54, 56). Each disc-shaped section (46, 48) has an interior face (60, 62) and an exterior face (64, 66).

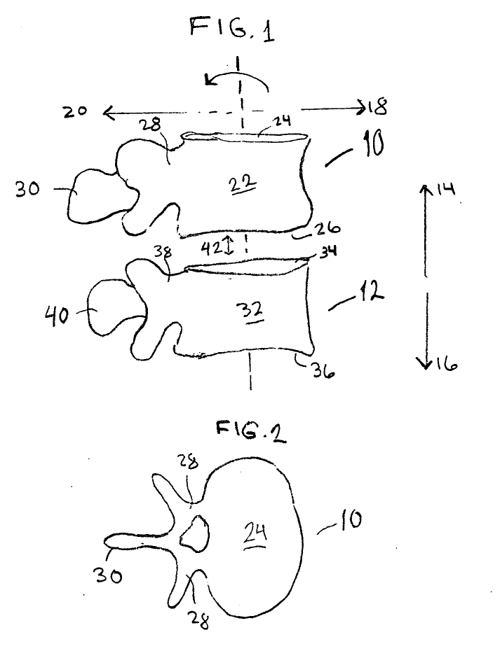

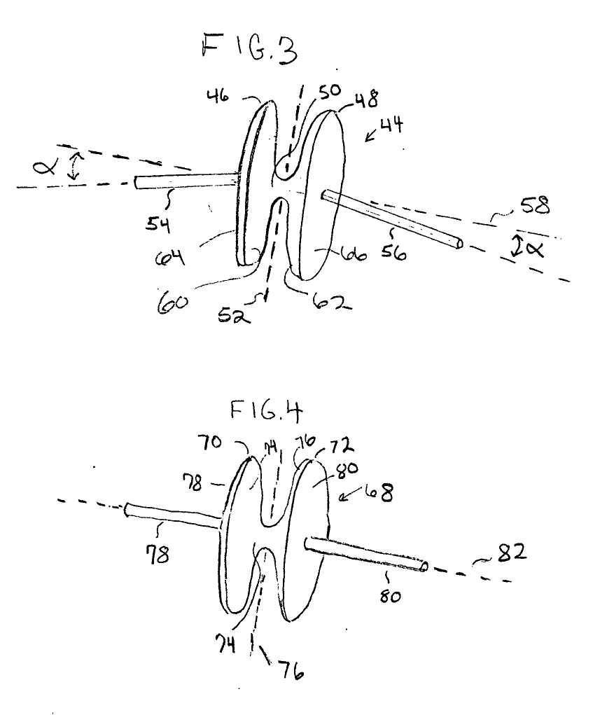

A second embodiment of the present invention is shown in FIG. 4, and comprises a body portion (68) having generally symmetrical and generally parallel disc-shaped sections (70, 72) joined by a middle portion (74) of a smaller diameter than the disc-shaped sections (70, 72) such that the middle portion (74) forms a generally horizontal axis when the disc-shaped portions (70, 72) are aligned with an imaginary vertical axis (76). A first arm (78) and a second arm (80) each extend from a respective one of said disc-shaped sections (70, 72) away from the center of the body portion (68) along an imaginary horizontal centerline (82) as shown by dotted line in FIG. 4. Each disc-shaped section (70, 72) has an interior face (74, 76) and an exterior face (78, 80).

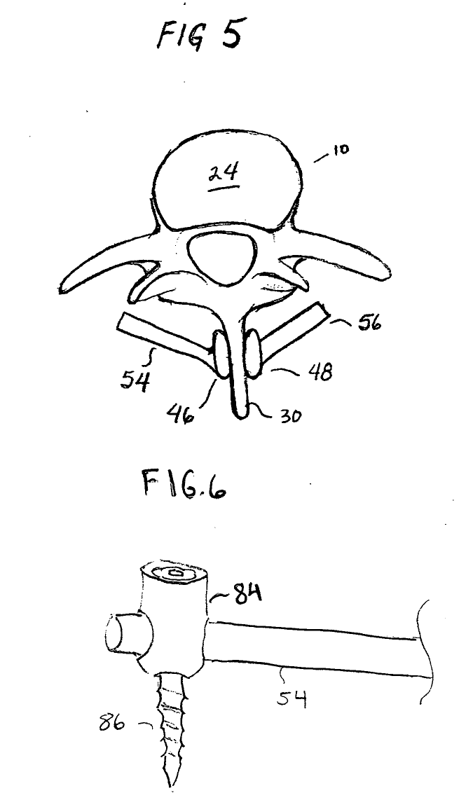

As shown in the diagrammatic view of FIG. 5, a device according to the first embodiment (FIG. 3) is inserted between vertebrae (10, 12) in a manner such that the center portion (50) is placed underneath the superior spinous process (30), supporting a portion of vertical load transmitted through the superior spinous process (30). While FIG. 5 shows the insertion of the first embodiment device, the second embodiment device (illustrated in FIG. 4) is inserted and held in essentially the same manner as shown in FIG. 5 and in the successive drawing figures.

FIG. 6 illustrates the end of arm (54) which preferably has a round diameter and is compatible with a screw retaining rod adapter (84) of any type generally known that fixes a pedicle screw (86) or other bone fastener to a rod-shaped member, in this case the arm (54). Such a rod adapter (84) is used in conjunction with and to facilitate application of the present invention. Other types of fastener adapters, including those that could adapt to the arm (54) if it were not of round cross-section, could be used to perform the same function. Alternatively, a bone-engaging fastener could be permanently attached to the arm (54) or integrally formed therewith (not shown).

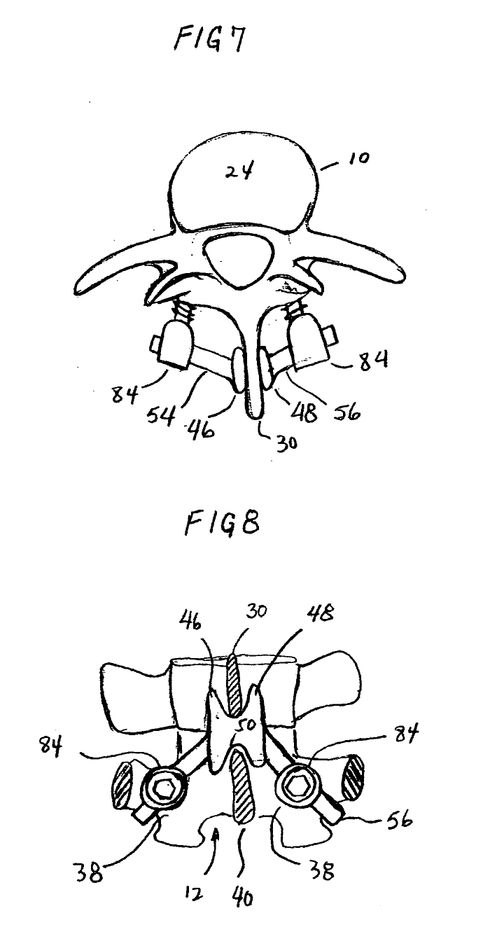

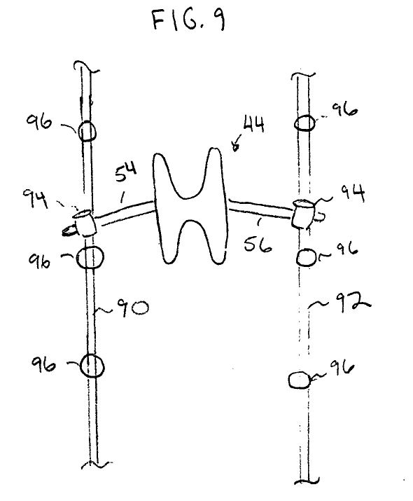

Referring to FIG. 7, the components as shown in FIG. 5 are illustrated with the addition of rod adapters (84) on each arm (54, 56) retaining pedicle screws thereon. As shown in FIG. 8, these pedicle screws (86) are fastened to the pedicle regions (38) of the inferior vertebra (12). The center portion (50) of the body (44) supports the superior spinous process (30) and transfers vertical load of the superior vertebra (10) through each arm (54, 56), through each rod adapter (84) and each pedicle screw (86), and finally to each pedicle region (38) of the inferior vertebra (12). The center portion (50) as well as any portion of the body (44) is not permitted to contact or transfer any vertical load to the inferior spinous process (40). Such an assembly applied as illustrated in FIGS. 7-8 is sized and configured to prevent over-rotation in the direction shown by arrow (88) in FIG. 1, and to distribute vertical load between the vertebrae (10, 12) during normal posture so that all the existing vertical load is not concentrated through the end faces (26, 34) via disc material or a disc prosthetic or implant.

As shown in FIG. 9, an alternative assembly using a device according to the embodiment of FIG. 3 comprises attaching the body (44) via the arms (54, 56) to first and second rods (90, 92) of a rod assembly of a type generally known in which rod couplers (94) of a type generally known are used to attach the arms (54, 56) to the rods (90, 92). The rods (90, 92) are attached as is generally known to vertebral bodies through bone fastener assemblies (96) of a type generally known, such as pedicle screws. The embodiment of FIG. 4 could also be implemented with the assembly of FIG. 9 in essentially the same manner as the embodiment of FIG. 3 is implemented.

The components of the assemblies according to the preferred embodiments may be made of any suitable, implantable material of sufficient strength and having additional characteristics suitable for such use. Such materials include but are not limited to surgical metals and alloys, composites and ceramics.

While the preferred embodiments of the present invention have been described, various modifications can be made without departing from the scope of the invention.

What is claimed is:

1) A device adapted to be inserted between two adjacent vertebrae, a first vertebra being a superior vertebra positioned above a second vertebra being an inferior vertebra, said device comprising
a body portion having a first side and a second side;
a central portion of said body separating said first and second sides;
a generally concave support surface on said central portion adapted to receive therein and support a portion of said superior vertebra; and
a pair of arms extending from said body portion in generally opposite directions away from said central portion and each being adapted to be fixed to said inferior vertebra.

2) A device according to claim 1, further comprising
a bone fastener adapter on a distal end of each arm; and
a bone fastener associated with each bone fastener adapter, each said bone fastener being adapted to be fixed to said inferior vertebra.

3) A device according to claim 1, wherein
said generally concave support surface is adapted to receive therein and support a lower portion of the superior process of said first vertebra.

4) A device according to claim 3, wherein
each said arm is adapted to be fixed to a pedicle region of said inferior vertebra.

5) An assembly for implanting between two adjacent vertebrae, a first vertebra being a superior vertebra positioned above a second vertebra being an inferior vertebra, said assembly comprising
a body portion having a first side and a second side;
a central portion of said body separating said first and second sides;
a generally concave support surface on said central portion adapted to received therein and support the spinous process portion of said superior vertebra;
a pair of arms extending from said body portion in generally opposite directions away from said central portion;
a pair of bone fastener adapters, each respectively attached to a distal end of one of said arms;
a pair of bone fasteners, each respectively attached to one of said bone fastener adapters and each respectively fastened to a portion of said inferior vertebra.

6) An assembly according to claim 5, wherein
each said portion of said inferior vertebra is a pedicle region of said inferior vertebra.

7) An assembly for implanting between two adjacent vertebrae, a first vertebra being a superior vertebra positioned above a second vertebra being an inferior vertebra, said assembly comprising
a body portion having a first side and a second side;
a central portion of said body separating said first and second sides;
a generally concave support surface on said central portion adapted to receive therein and support the spinous process portion of said superior vertebra;
a pair of arms extending from said body portion in generally opposite directions away from said central portion;
a pair of rod adapters, each respectively attached to a distal end of one of said arms;
a pair of rods generally parallel to each other and generally aligned with a vertical axis extending through said first vertebra and said second vertebra, each said rod being attached, respectively, to one of said rod adapters; and
a pair of bone fasteners, each respectively attached to one of said rods and each respectively fastened to a portion of said inferior vertebra.

8) An assembly according to claim 7, wherein
each said portion of said inferior vertebra is a pedicle region of said inferior vertebra.

9) A method of installing an assembly between two adjacent vertebrae, a first vertebra being a superior vertebra positioned above a second vertebra being an inferior vertebra, said method comprising
installing a body beneath and in contact with a lower surface of the spinous process of said superior vertebra; and
attaching said body to the pedicle region of said inferior vertebra.

10) A method according to claim 9, wherein
said body is attached to said pedicle region of said inferior vertebra by at least one pedicle screw.

11) A method according to claim 9, wherein
said body comprises a concave surface and said spinous process of said superior vertebra is received in contact with said concave surface.

12) A method according to claim 9, further comprising
providing a pair of arms extending from said body;
attaching a bone fastener to each arm; and
fastening each bone fastener to a pedicle region of said inferior vertebra.
